(19) United States
(12) Patent Application Publication
(10) Pub. No.: US 2012/0053542 A1
(43) Pub. Date: Mar. 1, 2012

(54) ACTUATOR FOR A NEGATIVE PRESSURE WOUND THERAPY SYSTEM

(75) Inventors: Jhy-Wen WU, TU-CHENG CITY (TW); Nan-Kuang YAO, TU-CHENG CITY (TW); Luo-Hwa MIAU, TU-CHENG CITY (TW); Jen-Chien CHIEN, TU-CHENG CITY (TW); Li-Ling LI, TU-CHENG CITY (TW)

(73) Assignee: APEX MEDICAL CORP., TU-CHENG CITY (TW)

(21) Appl. No.: 12/872,978

(22) Filed: Aug. 31, 2010

(51) Int. Cl. A61M 1/00

(52) U.S. Cl. 604/319

(57) ABSTRACT

An actuator has a pump head detachably connecting to an external motor. When the actuator extracts the pus and the infection subjects, the fluid only flows through the pump head so that only the pump head needs to be made of biocompatibility materials. Therefore, the manufacturing cost is reduced. Moreover, because the motor is not made of biocompatibility materials, only the pump head needs to be treated as medical wastes so that the disposal cost is reduced. Furthermore, since the pump head is detachable from the motor, the infected pump head is replaced independently to lower the replacement cost.

### BACKGROUND OF THE INVENTION

1. Field of the Invention

The present invention relates to an actuator, especially to an actuator for a negative pressure wound therapy system that creates a negative pressure environment in the wound and extracts pus.

2. Description of the Prior Arts

Negative pressure wound therapy utilizes wound sheets, soft suction pads, or biocompatibility pore materials to attach on the wounds and connects to a vacuum pump. The vacuum pump creates negative pressure in the wound to extract the pus and infection subjects and to draw the healthy tissue fluid so that a moist therapy environment is maintained. Therefore, the blood circulation around the wound is promoted to accelerate wound healing.

One of the conventional negative pressure wound therapy systems has a rigid collector connecting to a front end of the vacuum pump to extract the pus and the infection subjects into the rigid collector. A negative pressure sensor detects the negative pressure in the collector to determine whether the traditional system is operated normally. However, since the vacuum pump is connected to the rear end of the rigid collector, the pump is further from the wound so that the pump needs more power to create negative pressure in the wound and to extract the pus and the infection subjects from the wound.

Another conventional negative pressure wound therapy system solves the above problem. The collector is connected to the rear end of the vacuum pump. The vacuum pump is directly connected to the wound sheet attached on the wound so that the vacuum pump uses less power. Therefore, the pus and infection subjects unavoidably flows through the vacuum pump in the actuator. According to health care laws and in consideration of human healthy, all equipment directly contact human body and tissue fluid must be made of biocompatibility materials. However, the pump made of biocompatibility materials cost a lot. When the pump is broken, the pump also needs to be treated as a medical waste. The medical wastes have specialized treatment so that the pumps made of biocompatibility materials increase the disposal cost.

To overcome the shortcomings, the present invention provides an actuator for a negative pressure wound therapy system to mitigate or obviate the aforementioned problems.

### SUMMARY OF THE INVENTION

The main objective of the present invention is to provide an actuator for a negative pressure wound therapy system. The actuator has a pump head detachably connecting to an external motor. When the actuator extracts the pus and the infection subjects, the fluid only flows through the pump head so that only the pump head needs to be made of biocompatibility materials. Therefore, the manufacturing cost is reduced. Moreover, because the motor is not made of biocompatibility materials, only the pump head needs to be treated as medical wastes so that the disposal cost is reduced. Furthermore, since the pump head is detachable from the motor, the infected pump head is replaced independently to lower the replacement cost.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an actuator for a negative pressure wound therapy system in accordance with the present invention;

FIG. 2 is a partially perspective view of the actuator in FIG. 1;

FIG. 3 is an exploded perspective view of a negative pressure wound therapy system with the actuator in FIG. 1;

FIG. 4 is an exploded perspective view of the actuator in FIG. 1 with a sensor assembly;

FIG. 5 is a partially perspective view of another embodiment of an actuator for a negative pressure wound therapy system in accordance with the present invention;

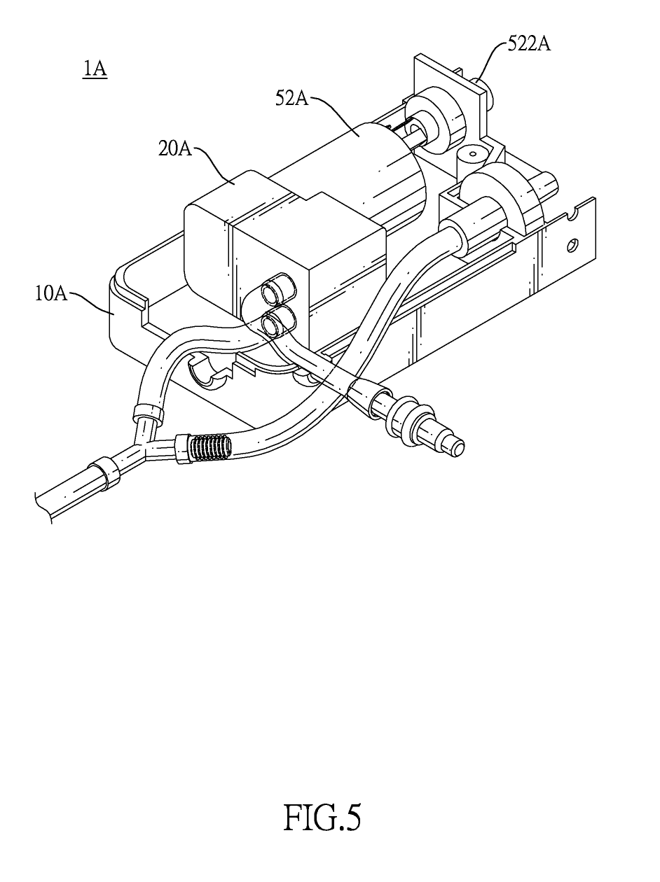

FIG. 6 is an exploded perspective view of the actuator in FIG. 5 with a sensor assembly; and

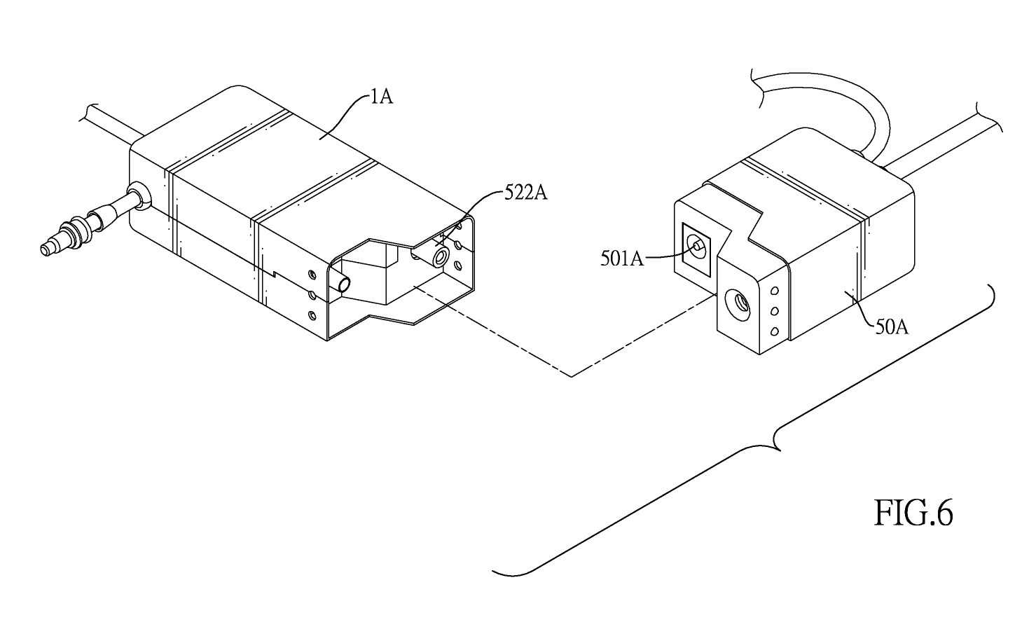

FIG. 7 is an exploded perspective view of a pump and a pump head of the actuator in FIG. 5.

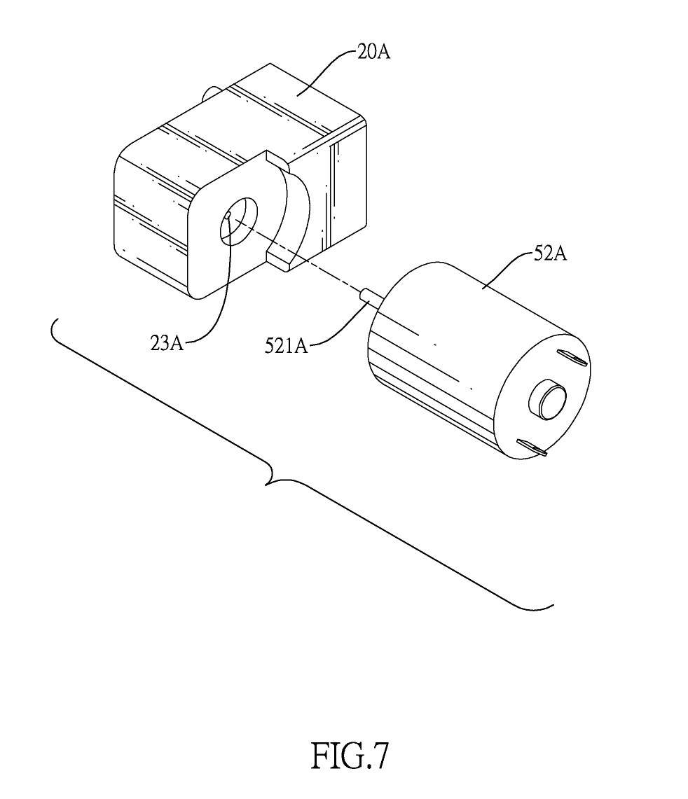

### DETAILED DESCRIPTION OF THE PREFERRED EMBODIMENTS

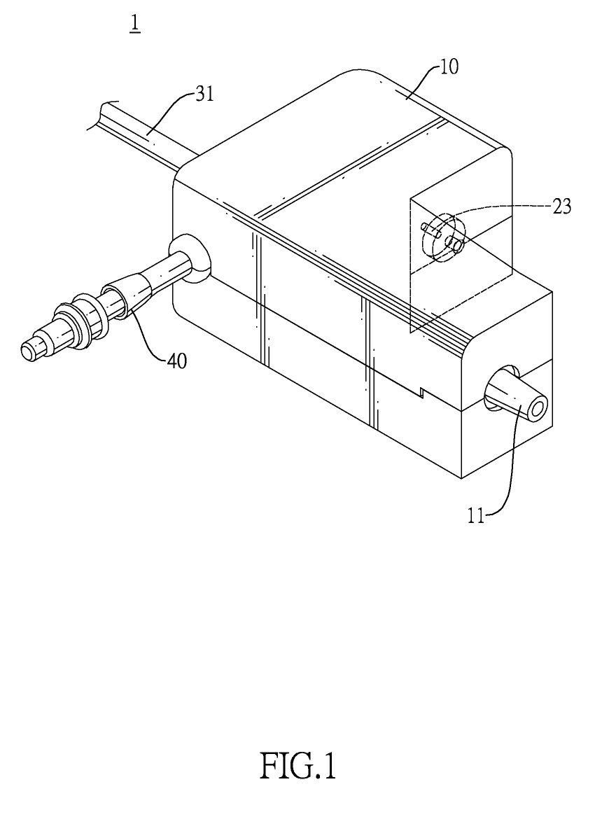

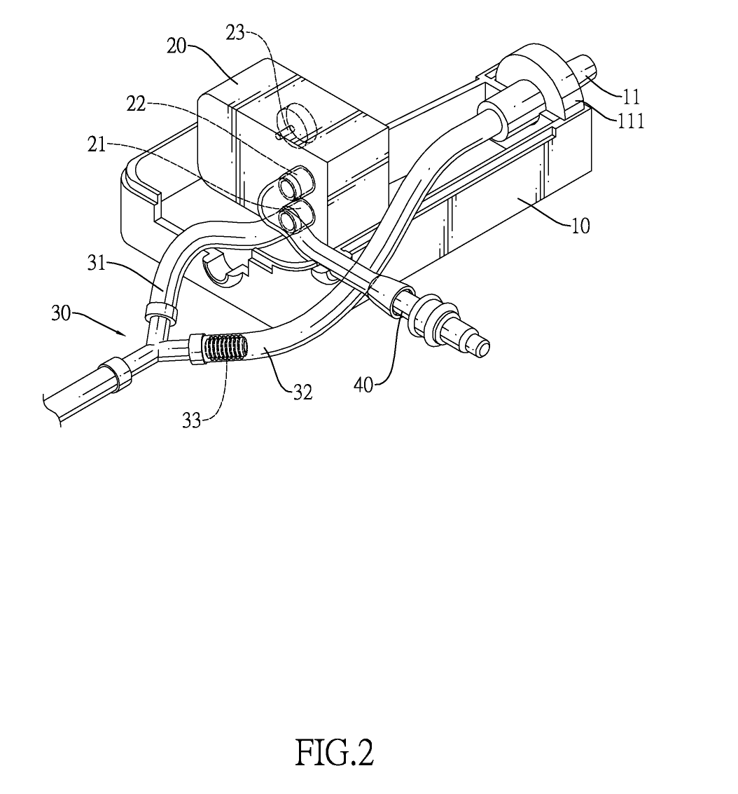

With reference to FIGS. 1 and 2, an actuator 1 for a negative pressure wound therapy system in accordance with the present invention comprises a shell 10, a pump head 20, an inlet connection 30 and an outlet tube 40.

The shell 10 is hollow and has a fluid connector 11 formed in a side thereof. The fluid connector 11 has a bisequesting unit 111 mounted therein to keep the infection subjects from flowing through the fluid connector 11.

With reference to FIG. 2, the pump head 20 is mounted in the shell 10 and is made of biocompatibility materials. The pump head 20 has two sides. An inlet connector 21 and an outlet connector 22 are formed on one side of the pump head 20. A driven connector 23 is formed on the other side of the pump head 20.

The inlet connection 30 connects between the fluid connector 11 and the pump head 20 and protrudes out of the shell 10. The inlet connection 30 may be a manifold with multiple passages. In a preferred embodiment, the inlet connection 30 has a first passage 31 and a second passage 32 communicating with each other. The first passage 31 connects to the inlet connector 21 of the pump head 20. The second passage 32 connects to the fluid connector 11.

The outlet tube 40 is connected to the outlet connector 22 of the pump head 20 and protrudes out of the shell 10.

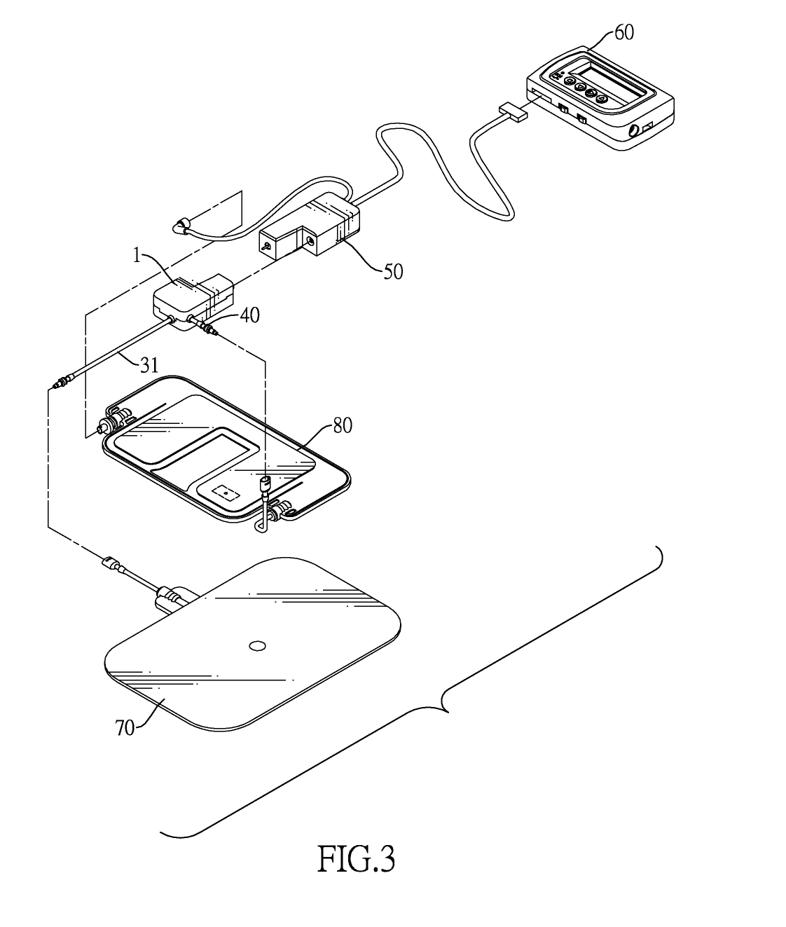

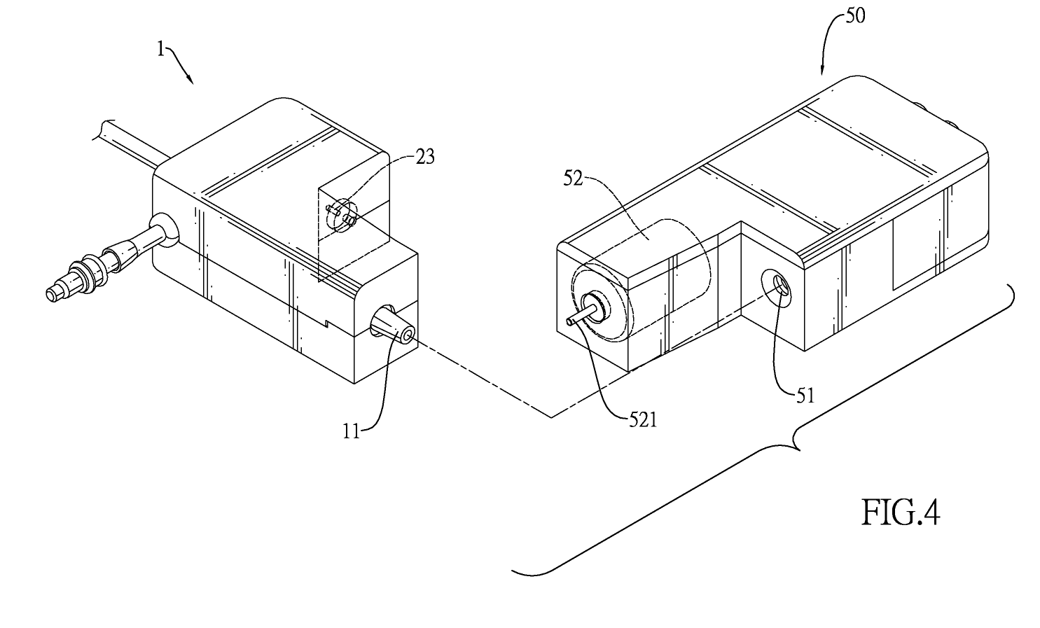

The actuator 1 is applied for a negative pressure wound therapy system. One of the negative pressure wound therapy system is shown in FIGS. 3 and 4. The actuator 1 is connected to the sensor assembly 50 via a fluid connector 51. The sensor assembly 50 has a motor 52 to drive the pump head 20. A transmission shaft 521 of the motor 52 is detachably inserted into the driven connector 23. The sensor assembly 50 is connected to the controller 60. The first passage 31 of the inlet connection 30 communicates with the wound through the wound-dressing unit 70. The outlet tube 40 connects to the collector 80.

With reference to FIGS. 2 to 4, the wound-dressing unit 70 is attached to the user's wound. When the negative pressure wound therapy system is started, the pump 52 drives the pump head 20 to create a negative environment in the wound and to extract pus and infection subjects. The pus and infection subjects pass through the first passage 31 and flows into the pump head 20, and then flows out of the pump head 20 through the outlet tube 40 and flows into the collector 80. Therefore, the pus and infection subjects only flows through the actuator 1 but does not flow through the motor 52. Since the motor 52 is detachable from the actuator 1, only the pump head 20, the inlet connection 30 and the outlet tube 40 need to be made of biocompatibility materials. Thus, the manufacturing cost is reduced. Moreover, the motor 52 is kept using while the infected actuator 1 is replaced so that the replacement cost is also reduced. When the motor 52 needs to be replaced, the broken motor 52 is treated as normal waste since the motor 52 does not contact the human tissue. Therefore, the disposal cost is also reduced.

Furthermore, the sensor assembly 50 detects the negative pressure in the wound through the fluid connectors 51, 11 and the second passage 32. When the pus and the infection subjects flow into the first passage 31, some of the pus and the infection subjects unavoidably flow into the second passage 32 since the first and second passages 31, 32 communicate with each other. However, the sensor assembly 50 will be damaged by the liquid so that a liquid limit is mounted in the second passage 32 to keep the liquid from entering the sensor assembly 50. In a preferred embodiment, the liquid limit is a spiral tube 33 with inner spiral wall. When the liquid flows into the second passage 32, the liquid flows along the spiral wall of the spiral tube 33 so that the flowing path is extended while the spiral tube 33 is short. Therefore, the liquid is blocked by the spiral tube 33 and does not damage the sensor assembly 50. The spiral tube 33 effectively extends the flowing path without increasing the length or volume.

In another preferred embodiment, the motor 52A is mounted in the shell 10A of the actuator 10A. The transmission shaft 521A of the motor 52A is connected detachably to the driven connector 23A of the pump head 20A. The motor 52A has an electrical connector 522A connecting to the electrical connector 501A of the sensor assembly 50A. The motor 52A is also detachable from the pump head 20A so that manufacturing cost and the replacement cost is reduced.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An actuator for a negative pressure wound therapy system comprising:
a shell having a fluid connector with a bisequesting unit mounted in the fluid connector;
a pump head made of biocompatibility materials, is mounted in the shell and has an outlet connector, an inlet connector and a driven connector;
an inlet connection respectively connecting to the inlet connector of the pump head and the fluid connector of the shell;
a liquid limit mounted in a part of the inlet connection, wherein the part of the inlet connection connects to the fluid connector of the shell; and
an outlet tube connecting to the outlet connector of the pump head.

2. The actuator as claimed in claim 1, wherein the liquid limit is a spiral tube with inner spiral wall.

3. The actuator as claimed in claim 1, wherein
the inlet connection is a manifold having a first passage and a second passage communicating with each other;
the inlet connector of the pump head connecting to the first passage;
the fluid connector of the shell connecting to the second passage; and
the liquid limit is mounted in the second passage.

4. The actuator as claimed in claim 2, wherein
the inlet connection is a manifold having a first passage and a second passage communicating with each other;
the inlet connector of the pump head connecting to the first passage;
the fluid connector of the shell connecting to the second passage; and
the spiral is inserted in the second passage.
